(19) United States
(12) Patent Application Publication
(10) Pub. No.: US 2024/0123064 A1
(43) Pub. Date: Apr. 18, 2024

(54) CUSTOM AUTOLOGOUS VACCINE COMPOSITION, AND A METHOD FOR ITS MANUFACTURE

(71) Applicant: Joseph CHALIFOUX, Nashua, NH (US)

(72) Inventor: Joseph CHALIFOUX, Nashua, NH (US)

(21) Appl. No.: 17/965,257

(22) Filed: Oct. 13, 2022

(51) Int. Cl. A61K 39/39; A61K 39/02

(52) U.S. Cl. CPC A61K 39/39; A61K 39/02; A61K 2039/53

(57) ABSTRACT

An immunogenic composition forming a vaccine includes an autologous cell medium, wherein producing the autologous cell medium further comprises producing the autologous cell medium using at least a call collected from a subject, therein the cell medium includes immune system stem cells, combining an oligonucleotide-based adjuvant with the autologous cell medium and combining an antigen with the autologous cell medium and the oligonucleotide-based adjuvant.

### FIELD OF THE INVENTION

The present invention generally relates to the field of vaccine compositions and methods of making and using the same. In particular, the present invention is directed to a custom autologous vaccine composition, and a method for its manufacture.

### BACKGROUND

Vaccinations (immunogenic compositions) work by activating the immune system and help the body recognize specific dangerous pathogens. Unfortunately, there are many people who have various adverse reactions to vaccines. The creation of custom vaccines which eliminate specific reactions and other adverse inflammatory, immunogenetic and/or idiosyncratic responses is an elusive goal. Therefore, there is a need for the creation of custom vaccines which eliminate these adverse reactions based on a foundation of bioidentical cell line constructs and unique adjuvants.

### SUMMARY OF THE DISCLOSURE

In an aspect, an immunogenic composition forming a vaccine includes an autologous cell medium, wherein producing the autologous cell medium further comprises producing the autologous cell medium using at least a cell collected from a subject, wherein the cell medium includes immune system stein cells, combining an oligonucleotide-based adjuvant with the autologous cell medium and combining an antigen with the autologous cell medium and the oligonucleotide-based adjuvant.

In another aspect, a method of manufacturing an immunogenic composition forming a vaccine includes receiving an autologous cell medium, wherein producing the autologous cell medium further comprises producing the autologous cell medium using at least a cell collected from a subject, therein the cell medium includes immune system stem cells, combining an oligonucleotide-based adjuvant with the autologous cell medium and combining an antigen with the autologous cell medium and the oligonucleotide-based adjuvant.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an immunogenic composition;

FIG. 2 is block diagram illustrating an exemplary embodiment of ingredients contained within the immunogenic composition.

FIG. 3 is a diagram of an exemplary embodiment of a machine-learning module;

FIG. 4 is a diagram of an exemplary embodiment of a neural network;

FIG. 5 is a diagram of an exemplary embodiment of a node of a neural network;

FIG. 6 is a diagram of an exemplary embodiment of a fuzzy set comparison;

FIG. 7 is a block diagram illustrating an exemplary method of manufacturing an immunogenic composition; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Embodiments disclosed herein present a novel custom vaccine designed by using one or more cells collected from the donor. A resulting vaccine may be scalable, flexible in its antigen presentation, and have the potential for stability outside the cold chain. In an embodiment, a vaccine may include a positively charged chemical vaccine additive for cell targeting and may include a unique adjuvant and an antigen. A customized vaccine schedule based on donor's own cell lines and unique adjuvant may be generated for adults and children and may be further customized for immunocompromised subjects. The customized vaccine schedule may be generated by a machine-learning model.

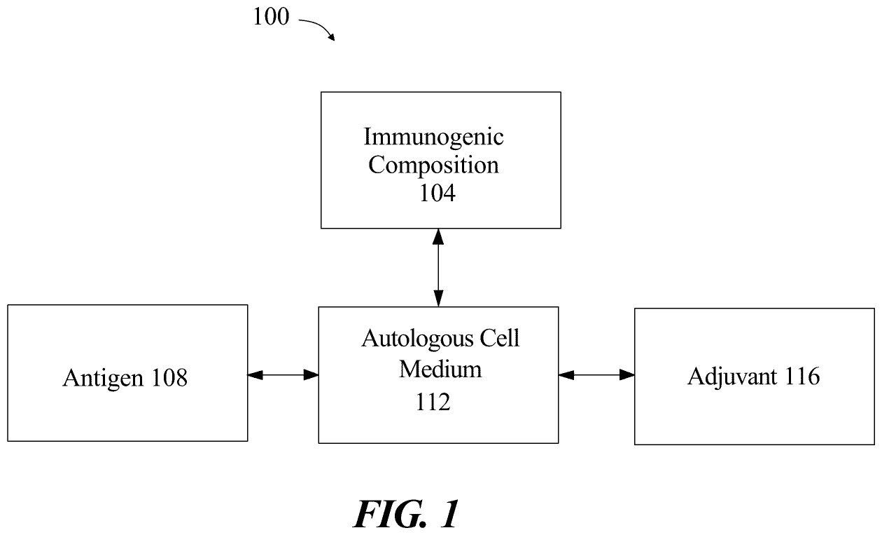

Referring now to FIG. 1, an exemplary embodiment of an immunogenic composition 100 is illustrated. An autologous cell medium is received, wherein producing the autologous cell medium further comprises producing the autologous cell medium using at least a cell collected from a subject, wherein the cell medium includes immune system stem cells. Immunogenic (vaccine) composition 104 may be a synthesized inactivated vaccine, live-attenuated vaccine, messenger RNA (mRNA) vaccine, subunit vaccine, recombinant vaccine, polysaccharide vaccine, conjugate vaccine, toxoid vaccine and viral vector vaccine and the like. Immunogenic (vaccine) composition 104 may comprise an autologous cell medium 112. A “cell medium” as used in this disclosure is defined as a liquid or gel designed to support the growth of cells or microorganisms. “Autologous” as used in this disclosure is defined as derived from the same individual. Cell medium 112 may include immune system stem cells such as fibroblast cells, retinal cells, neutrophils, eosinophils, basophils, mast cells, monocytes, macrophages, dendritic cells, natural killer cells, and lymphocytes (B cells and T cells). Autologous cell medium 112 may include one or more cells collected from a donor. The donor may be the person receiving the vaccine that is produced using the donor's own cells to grow the vaccine. This may reduce the risk of adverse events typically seen when animal cells are used to grow and produce vaccines. In some embodiments, immune stem cells may be extracted from a user and stored for providing the custom vaccine based on a personalized vaccine schedule of the user. For example, the umbilical cord of an infant may be stored, wherein the stem cells are extracted and used as cell lines in producing a plurality of customized bio-identical vaccines based on the child's growth. In an embodiment, autologous cell medium 112 may be stored and/or preserved for future research purposes such as in a library and/or index of genetic material. In another embodiment, pluripotent cells may be used. “Pluripotent” as used in this disclosure is defined as an immature or stem cell capable of giving rise to several different cell types. For example, many types of cells originate from pluripotent bone marrow stem cells. In cases where an autologous cell line is not possible, an allogenic cell line may be used. “Allogenic” as used in this disclosure is defined as human cells derived from a donor other than the patient, where the donor is genetically non-identical. For example, the stem cells in allogenic transplants are from a person other than the patient, either a matched unrelated donor or a matched related donor. In an embodiment, a subject's own cells may be used with a cell line being produced from them which is then modified. For example, a genetically modified cell line may be produced by using human stem cell lines via the introduction of nucleases and/or foreign genetic material leading to stable genomic alteration. In another embodiment, a subject's cells may be altered so they are no longer human cells. In another embodiment, the medium may be extracted from the cell line. The resulting composition is transformed into a vaccine delivery medium which no longer comprises viable human cells. In an embodiment, the cell lines may be stored in a warehouse or storage facility utilizing, for example, cryopreservation. “Cryopreservation” as used in this disclosure is defined as a method whereby cells are frozen, maintaining their viability, until they are defrosted after a period of time. Cells are cryopreserved to minimize genetic change and avoid loss through contamination. A warehouse or storage facility may also be utilized for the growing of cell lines. For example, a cell line may be grown by using DMEM (Dulbecco's Modified Eagle Medium) culture media or RPMI (Roswell Park Memorial Institute Medium) culture media with FBS (Fetal Bovine Serum), 2 mM glutamine and antibiotics if required. In an embodiment, cell lines may be digitally or electronically stored. For example, cell lines may be manufactured by essentially fabricating cells, a subject's genetic data may be digitally stored and then the type of cell desired to be created may be fabricated or printed with immunogenic composition 104 being manufactured from this type of cell line. Alternatively or additionally immunogenic composition 104 may be manufactured from the unique subject data directly. Immunogenic composition 104 may be manufactured using machine learning. Immunogenic composition 104 may be manufactured using automation including but not limited to the use of robotics, artificial intelligence, compliance automation, customer operation automation, automating inspection systems, additive manufacturing and the like.

Immunogenic composition 104 is further manufactured by combining an oligonucleotide-based adjuvant 116 with the autologous cell medium. An “adjuvant,” as used in this disclosure, is a pharmacological and/or immunological agent that improves, or helps to stimulate, an immune response of a vaccine, antigen, or other immunologically active compound. Adjuvant 116 may be oligonucleotide-based. “Oligonucleotide” as used in this disclosure is a short DNA or RNA molecule or oligomer. Oligonucleotides serve as the starting point for many research, genetic testing and forensics applications. In some embodiments, an oligonucleotide-based adjuvant may include an antisense oligonucleotide, hexamer oligonucleotide, CpG oligonucleotide, and/or other forms of oligonucleotides. In some embodiments an oligonucleotide-based adjuvant may be received from the user and purified in the cell line. In some embodiments, the vaccine adjuvant 116 may contain a non-toxic ingredient such as silver.

Immunogenic composition 104 may also include synthetization with improved aluminum adjuvants, polyacrylic acid polymer adjuvants stabilized silver nanoparticles, silver nanorods, Iron nanoparticles, Saponins (amphipathic glycosides), Triterpenoid Saponins, Tomatine, Plant Polysaccharide Adjuvant Inulin, Mushroom Polysaccharides (β-1,3-D-gluco-pyanans with β-1,6-d-glucosyl branches, proteoglycan), Ganoderma, heteroglycan, mannoglycan, glycoprotein, Lentinan, glucan, mannoglucan, proteoglycan, Acidic polysaccharide, Endophytic Fungi, Marine Sponge α-GalCer, Marine Crustacean Chitosan, Propolis Compounds), Bee Venom, and existing (US) adjuvants such as amorphous aluminum hydroxyphosphate sulfate (AAHS), aluminum hydroxide, aluminum phosphate, potassium aluminum sulfate (Alum), Monophosphoryl lipid A (MPL)+aluminum salt (“AS04”), Oil in water emulsion composed of squalene (“MF59”), Monophosphoryl lipid A (MPL) and QS-21, a natural compound extracted from the Chilean soapbark tree, combined in a liposomal formulation (“AS01”), existing Cytosine phosphoguanine (CpG), a synthetic form of DNA that mimics bacterial and viral genetic material (“CpG 1018”). In some embodiments adjuvant may include organic or other enhanced adjuvants such as nano silver, organic silver, charged silver, liposomal silver and the like which are calculated for lowest individual and group and/or sex risk and efficacy and the like.

Immunogenic composition 104 is further manufactured by combining an antigen with the autologous cell medium and the oligonucleotide-based adjuvant. An “antigen,” as used in this disclosure, is a viral molecule and/or molecular structure that may induce an antigen-specific antibody response and/or result in immune cell antigen receptor-binding. In an embodiment, antigen 108 may contain a weakened (attenuated) and/or inactivated form of a virus or bacterium. For example, the measles vaccine is an attenuated live virus vaccine whereby after injection, the viruses cause a harmless infection in the vaccinated person with few symptoms before they are eliminated from the body. The antigen 108 may include a protein fragment of a virus or bacterium. Protein-based antigens are advantageous because though different molecules can serve as antigens, only proteins are capable of inducing both cellular and humoral immunity. The antigen 108 may include mRNA. For example, nucleic acid vaccines containing antigens encoded by RNA may be delivered through the use of a viral vector, like an adenovirus, or through the use of a non-viral delivery system such as electroporation. The two general classes of mRNA's that are commonly used as vaccine genetic vectors within RNA vaccines are self-amplifying mRNA and non-replicating mRNA. Non-replicating mRNA only encodes protein antigens of interest and self-amplifying mRNA encodes proteins allowing for RNA replication. The antigen 108 may include a vector such as a carrier virus that has been modified. The vector virus delivers important instructions to the patient's cells on how to recognize and fight the virus that causes the underlying disease (e.g., COVID-19). For example, some COVID-19 vaccines use a vector virus. The immunogenic composition manufactured by the steps described herein may also be utilized to manufacture various medicines on a custom, bioidentical basis.

The immunogenic composition 104 may be integrated with a “targeting system” of the delivery of the custom immunogenic composition (vaccine) to the specific receptor sites to further elevate the precision model of therapy. The targeting system may be similar to CAR-T cell therapy. “CAR-T cell therapy” (Chimeric Antigen Receptor) as used in this disclosure is defined as a type of treatment in which a patient's T cells (type of immune system cell) are engineered so they directly attack the cells infected by pathogens. For example, the chimeric antigen receptor (CAR) T cells are T cells which are genetically engineered to produce an artificial T cell receptor for use in immunotherapy. In an embodiment, the patient's cells may be altered in order to proliferate in response to the adjuvant and calculated in anticipation of the viral vector(s) introduced by the vaccine. The gene for the target receptor that binds to a certain protein on the patient's specific/target receptor (a chimeric antigen receptor, or “CAR”) cells would be added to the T cells in a laboratory. These custom T cells may then be proliferated and, depending on the number and/or volume, may be included in the vaccine dose or potentially require infusion. This would, instead of the traditional ‘passive’ or rather ‘downstream’ adjuvant method where T cells are stimulated by adjuvant excitement with the vaccine injection to further create an antibody creation response, result in custom T cells injected and/or infused with the vaccine to more precisely dictate the sought immune response.

Still referring to FIG. 1, vaccine may be administered in any suitable manner. In an embodiment, vaccine may be injectable. Vaccine may alternatively or additionally be absorbed through a mucous membrane, for instance via aerosolized delivery to the nostrils and/or lungs. Alternatively or additionally, vaccine may be administered using a patch, such as without limitation a microneedle patch that delivers lyophilized vaccine in powder form; as a non-limiting example, lyophilized vaccine may be included in soluble microneedles which upon insertion to tissue of a living organism may dissolve in fluids thereof, reconstituting and activating the vaccine. As a further non-limiting example, lyophilized vaccine may be delivered in an implant such as a soluble or insoluble needle inserted under the skin or into other tissue allowing fluids of the subject tissue to reconstitute and disseminate the vaccine. Vaccine may be delivered in liquid and/or lyophilized form to any mucous membrane; for instance, and without limitation, vaccine may be delivered as a lyophilized inhalable powder for absorption in nasal and/or pulmonary surfaces. Vaccine may be delivered orally, for instance in a needle or other device for injecting lyophilized vaccine into and/or across digestive tissues, which may be delivered in a capsule designed to disintegrate in one or more digestive juices. Vaccine in lyophilized form may be delivered by a nanobot.

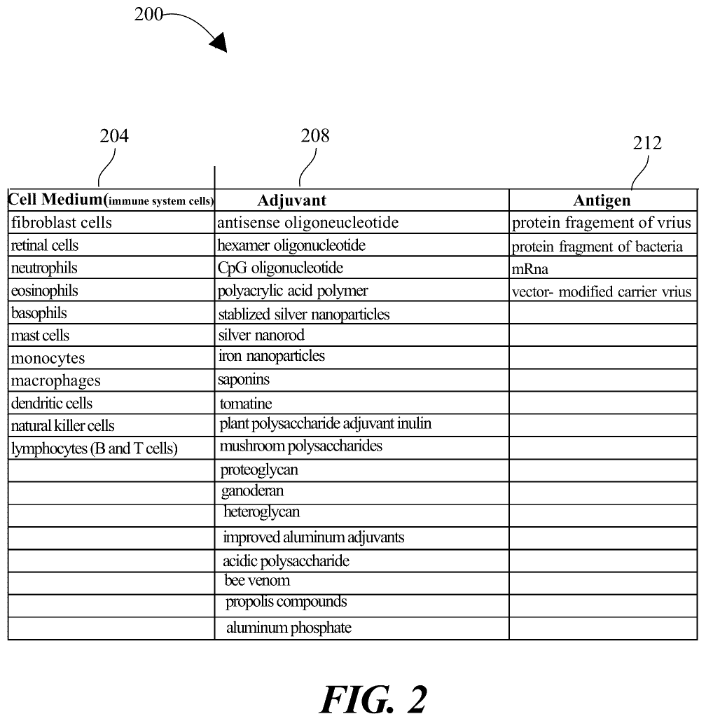

Referring now to FIG. 2, an exemplary embodiment of ingredients 200 contained within formulation 100 is illustrated. Formulation 100 may include a cell medium 112, wherein composition 104 includes any of the compositions 104 described above, in reference to FIG. 1, and wherein composition 104 may include but is not limited to any of the compositions contained within column 204. Formulation 100 may include an adjuvant 108, wherein adjuvant 108 includes any of the adjuvants described above, in reference to FIG. 1, and wherein adjuvant 108 may include but is not limited to any of the adjuvants 108 contained within column 208. Formulation 100 may include an antigen 112, wherein antigen 112 includes any of the antigens 112 described above, in reference to FIG. 1, and wherein antigen 112 may include but is not limited to any of the antigens 112 contained within column 212.

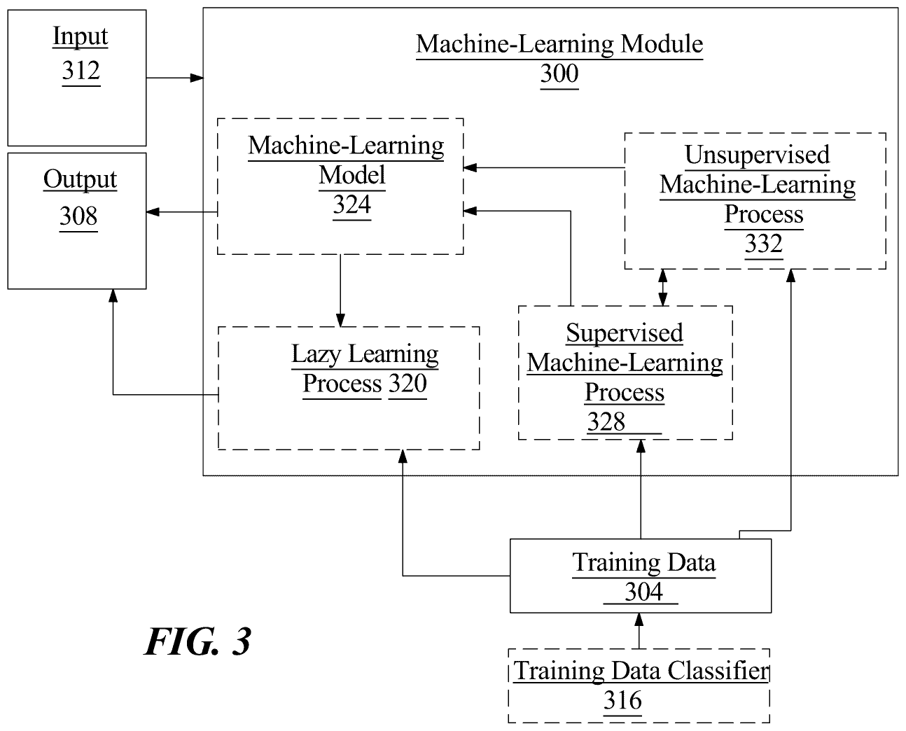

A custom vaccine schedule may be generated. Customizing a vaccine schedule may include customizing the type of vaccine and time of administration. For example, a child receiving a DTaP shot at 2 months and then receiving a booster shot at 11 years. In some embodiments, customizing the vaccine schedule may also include customizing the vaccine dosing in a person. In some embodiments, customization may include the type of vaccine administration, such as oral and mucosal vaccination. In some embodiments, a vaccine schedule may be customized to a user based on biological factors such as age, sex, genetics, comorbidities, immune response, and the like. For example, in adults, a vaccine schedule may follow the average vaccine schedule for adults 18 years and older. Furthermore, the vaccine schedule may be customized for adults during outbreaks of viruses such as COVID-19. In infants and children, a vaccine schedule may be customized based on their growth. The child vaccine schedule may be customized based on parental/guardian wishes to space out vaccinations throughout childhood. The vaccine schedule may be generated using a machine-learning model as discussed below and in reference to FIG. 3.

The vaccine schedule may be customized for immunocompromised users. “Immunocompromised” as used in this disclosure is defined as having an impaired immune system. This may be diagnosed by obtaining immunity health data of the user by, for example, obtaining an analysis of such as the user's blood. For example, a blood test may show the user's white blood cell count. White cells in the blood are an integral part of the human immune system and when a person gets sick the body produces more white blood cells to fight the viruses, bacteria or other pathogens causing the illness, therefore, if a person's white blood cell count is abnormally high this may indicate an immunocompromised condition. Immunity health data may be utilized to determine a degree of immunodeficiency of the user. For example, this may be performed by data classifying a subject to be immunocompromised. A processor and/or computing device may utilize a machine learning processes to conduct the comparison of user and immunity health data inputs. In some embodiments, a machine learning algorithm input may be the plurality of user inputs, wherein the training data may be the inputs of immunity health data XX, and the algorithm output may be the degree of immunodeficiency.

Additionally, or alternatively, processor and/or computing device may utilize a knowledge-based system (KBS) to compare inputs for compatibility. As used in this disclosure, a KBS is a computer program that reasons and uses a knowledge base to solve complex problems. The KBS has two distinguishing features: a knowledge base and an inference engine. A knowledge base may include technology used to store complex structured and unstructured information used by a computer system, often in some form of subsumption ontology rather than implicitly embedded in procedural code. Other common approaches in addition to a subsumption ontology include frames, conceptual graphs, and logical assertions. In some embodiments, the knowledge base may be a storage hub that contains information about past matches of users to postings based on the similarity of inputs and feedback from users and employers about the compatibility of matches. Next, an Inference engine allows new knowledge to be inferred. For example, the inference engine may determine that a user is associated more often with a high degree of immunodeficiency when the user input includes “White Blood Cell Count”+“C-Reactive Protein Count” rather than just the “White Blood Cell Count” alone. Most commonly, it can take the form of IF-THEN rules coupled with forward chaining or backward chaining approaches. Forward chaining starts with the known facts and asserts new facts. Backward chaining starts with goals and works backward to determine what facts must be asserted so that the goals can be achieved. Other approaches include the use of automated theorem provers, logic programming, blackboard systems, and term rewriting systems such as CHR (Constraint Handling Rules). The inference engine may make predictions or decisions in optimizing classifying postings to a user without being explicitly programmed to do so. The inference engine may receive constant feedback and self-learn based on previous classifications, as described through this disclosure, and recommendations to further refine and strengthen its recommendations.

Processor and/or computing device may be configured to classify the user's degree of immunodeficiency as a function of the comparison. Classifier may include a classification algorithm wherein the algorithm output is a degree of immunodeficiency optimized for the user. In some embodiments, the classification algorithm may take a plurality of user inputs as inputs, wherein the training data includes a plurality of immune health data inputs, data from a KBS, output data of any other classification/comparison described throughout this disclosure, and the like.

Processor and/or computing device, as a function of the comparison, may be configured to rank a plurality of degrees of immunodeficiency in order of similarity to a user immune health data, wherein a rank of degrees of immunodeficiency is based on the similarity score. In some embodiments, generating the ranking may include linear regression techniques. Processor and/or computing device may be designed and configured to create a machine-learning module using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Processor and/or computing device may be configured to use classifier to classify, as a function of ranking, the user to a ranked plurality of degrees of immunodeficiency. In some embodiments, processor and/or computing device may be configured to produce classification output results including the classified ranked postings in a selectable format by user. For example, user may select to output classified ranked postings in a pie chart, wherein the ranked classified postings are divided, and color coded in selectable classification bins. This may be any classifier as described in further detail below.

The vaccine schedule may be generated using a machine-learning model. Any and all determinations described above may be performed and analyzed using an optimization program. Processor may compute a score associated with the threshold and select compliance items to minimize and/or maximize the score, depending on whether an optimal result is represented, respectively, by a minimal and/or maximal score; a mathematical function, described herein as an “objective function,” may be used by processor to score each possible pairing. Objective function may be based on one or more objectives as described below. Each factor may be assigned a score based on predetermined variables. In some embodiments, the assigned scores may be weighted or unweighted.

Processor may generate an objective function. An “objective function” as used in this disclosure is a process of minimizing or maximizing one or more values based on a set of constraints. In some embodiments, an objective function of apparatus 100 may include an optimization criterion. For example, an optimization criterion may be a threshold. An optimization criterion may include any description of a desired value or range of values for one or more attributes; desired value or range of values may include a maximal or minimal value, a range between maximal or minimal values, or an instruction to maximize or minimize an attribute. As a non-limiting example, an optimization criterion may specify that an attribute should be within a 1% difference of an attribute criterion. An optimization criterion may alternatively request that an attribute be greater than a certain value. An optimization criterion may specify one or more tolerances for precision in a matching of attributes to improvement thresholds. An optimization criterion may specify one or more desired attribute criteria for a matching process. In an embodiment, an optimization criterion may assign weights to different attributes or values associated with attributes. One or more weights may be expressions of value to a user of a particular outcome, attribute value, or other facet of a matching process. Optimization criteria may be combined in weighted or unweighted combinations into a function reflecting an overall outcome desired by a user; function may be an attribute function to be minimized and/or maximized. A function may be defined by reference to attribute criteria constraints and/or weighted aggregation thereof as provided by processor; for instance, an attribute function combining optimization criteria may seek to minimize or maximize a function of improvement threshold matching.

Optimizing an objective function may include minimizing a loss function, where a “loss function” is an expression an output of which an optimization algorithm minimizes to generate an optimal result. As a non-limiting example, processor may assign variables relating to a set of parameters, which may correspond to score attributes as described above, calculate an output of mathematical expression using the variables, and select a pairing that produces an output having the lowest size, according to a given definition of “size,” of the set of outputs representing each of plurality of candidate improvement thresholds; size may, for instance, included absolute value, numerical size, or the like. Selection of different loss functions may result in identification of different potential pairings as generating minimal outputs. Objectives represented in an objective function and/or loss function may include minimization of differences between attributes and improvement thresholds.

Optimization of objective function may include performing a greedy algorithm process. A “greedy algorithm” is defined as an algorithm that selects locally optimal choices, which may or may not generate a globally optimal solution. For instance, processor may select immune health data so that scores associated therewith are the best score for vaccine schedule considering a user who is immunodeficient.

Objective function may be formulated as a linear objective function, which processor may solve using a linear program such as without limitation a mixed-integer program. A “linear program,” as used in this disclosure, is a program that optimizes a linear objective function, given at least immune health data of a user. A mathematical solver may be implemented to solve for the set construction and geographical constraints that maximizes scores; mathematical solver may be implemented on a processor and/or another device, and/or may be implemented on third-party solver.

Optimizing objective function may include minimizing a loss function, where a “loss function” is an expression an output of which an optimization algorithm minimizes to generate an optimal result. As a non-limiting example, processor may assign variables relating to a set of parameters, which may correspond to score components as described above, calculate an output of mathematical expression using the variables, and select a construction constraint that produces an output having the lowest size, according to a given definition of “size,” of the set of outputs representing each of plurality of candidate ingredient combinations; size may, for instance, included absolute value, numerical size, or the like. Selection of different loss functions may result in identification of different potential pairings as generating minimal outputs.

Referring now to FIG. 3, a diagram of an exemplary embodiment of a machine-learning module is presented. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 300 may generate a classifier using a classification algorithm, defined as a process whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs as described above in this disclosure, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Fresenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

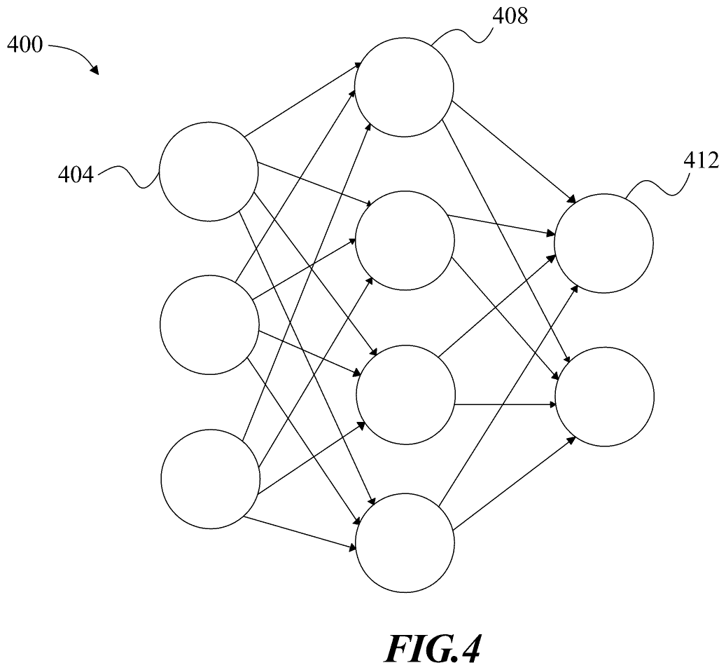

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

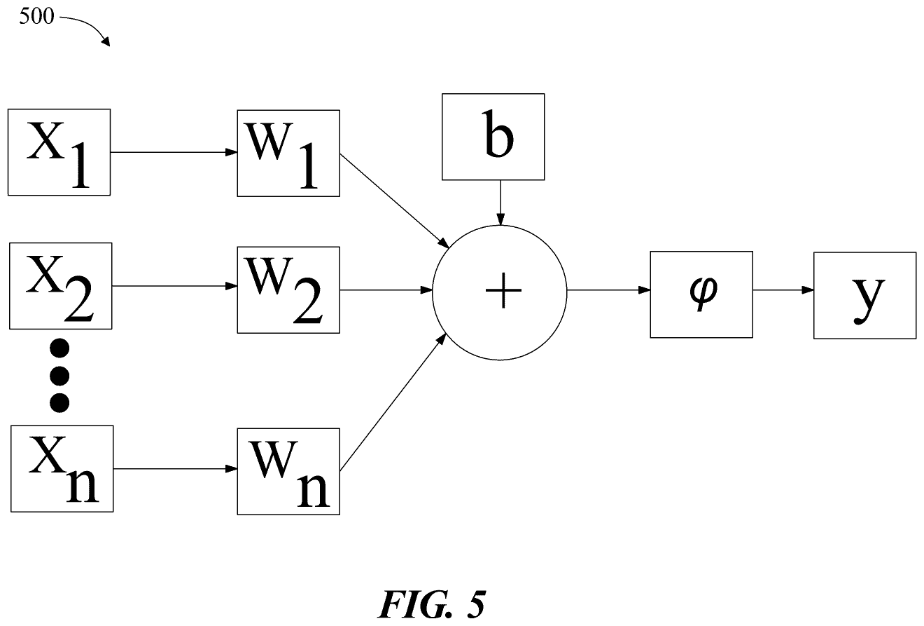

Referring now to FIG. 5, an exemplary embodiment 500 of a node of a neural network is illustrated. A node may include, without limitation, a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

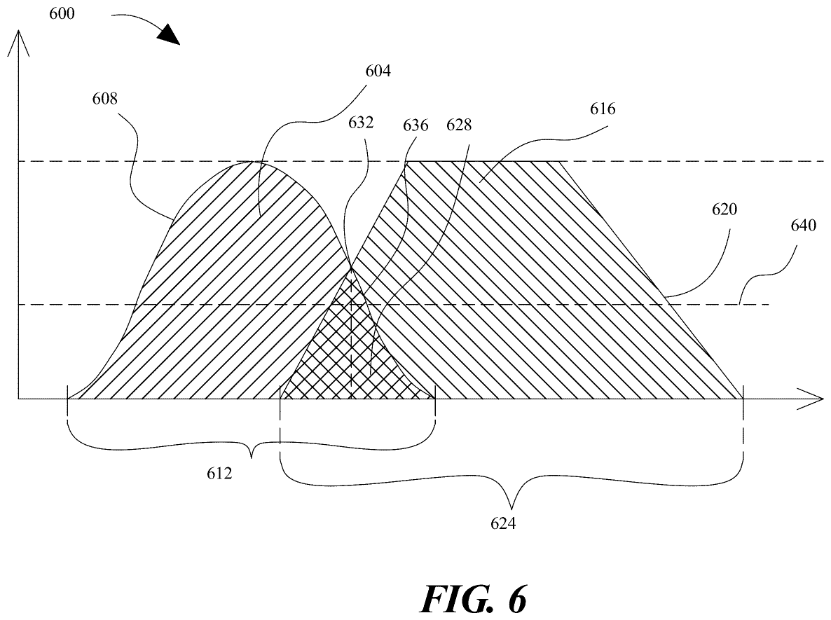

Referring to FIG. 6, an exemplary embodiment of fuzzy set comparison 600 is illustrated. A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 804. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , & for x > c and x < a \\ \frac{x - a}{b - a} , & for a \leq x < b \\ \frac{c - x}{c - b} , & if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 6, first fuzzy set 604 may represent any value or combination of values as described above, including output from one or more machine-learning models. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, threshold may indicate a sufficient degree of overlap between an output from one or more machine-learning models and a predetermined class, for combination to occur as described above. Alternatively or additionally, each threshold may be tuned by a machine-learning and/or statistical process, for instance and without limitation as described in further detail below.

Further referring to FIG. 6, in an embodiment, a degree of match between fuzzy sets may be used to determine a custom vaccine schedule based on input data such as user input data. Where multiple fuzzy matches are performed, degrees of match for each respective fuzzy set may be computed and aggregated through, for instance, addition, averaging, or the like, to determine an overall degree of match.

Still referring to FIG. 6, in an embodiment, custom vaccine schedule may be compared to multiple immune health data fuzzy sets. Machine-learning methods as described throughout may, in a non-limiting example, generate coefficients used in fuzzy set equations as described above, such as without limitation x, c, and σ of a Gaussian set as described above, as outputs of machine-learning methods. Likewise, a custom vaccine schedule may be used indirectly to determine a fuzzy set, as custom vaccine schedule fuzzy set may be derived from outputs of one or more machine-learning models that take the user health data directly or indirectly as inputs.

Still referring to FIG. 6, a computing device may use a logic comparison program, such as, but not limited to, a fuzzy logic model to determine a score. A score may include, but is not limited to, amateur, average, knowledgeable, superior, and the like; each such score may be represented as a value for a linguistic variable representing score, or in other words a fuzzy set as described above that corresponds to a degree of similarity as calculated using any statistical, machine-learning, or other method that may occur to a person skilled in the art upon reviewing the entirety of this disclosure. In other words, a given element of a custom vaccine schedule may have a first non-zero value for membership in a first linguistic variable value and a second non-zero value for membership in a second linguistic variable value. In some embodiments, determining a score may include using a linear regression model. A linear regression model may include a machine learning model. A linear regression model may be configured to map data of design compliance plans to one or more scores. A score classification model may be configured to input collected data and cluster data to a centroid based on, but not limited to, frequency of appearance, and the like. Centroids may include scores assigned to them such that elements of the design plan may each be assigned a score. In some embodiments, and score classification model may include a K-means clustering model. In some embodiments, and score classification model may include a particle swarm optimization model. In some embodiments, determining a score of immunodeficiency may include using a fuzzy inference engine. In some embodiments, a plurality of entity assessment devices may be arranged by a logic comparison program into score arrangements. An “score arrangement” as used in this disclosure is any grouping of objects and/or data based on skill level and/or output score. This step may be implemented as described above in FIGS. 1-7. Membership function coefficients and/or constants as described above may be tuned according to classification and/or clustering algorithms. For instance, and without limitation, a clustering algorithm may determine a Gaussian or other distribution of questions about a centroid corresponding to a given score level, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Further referring to FIG. 6, an inference engine may be implemented according to input and/or output membership functions and/or linguistic variables. For instance, a first linguistic variable may represent a first measurable value pertaining to a degree of similarity, while a second membership function may indicate a degree of similarity of a subject thereof, or another measurable value. Continuing the example, an output linguistic variable may represent, without limitation, a score value. An inference engine may combine rules, such as: “if the difficulty level is ‘hard’ and the popularity level is ‘high’, the question score is ‘high’”—the degree to which a given input function membership matches a given rule may be determined by a triangular norm or “T-norm” of the rule or output membership function with the input membership function, such as min (a, b), product of a and b, drastic product of a and b, Hamachi product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥,” such as max(a, b), probabilistic sum of a and b (alba*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugano-King (TSK) fuzzy model.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

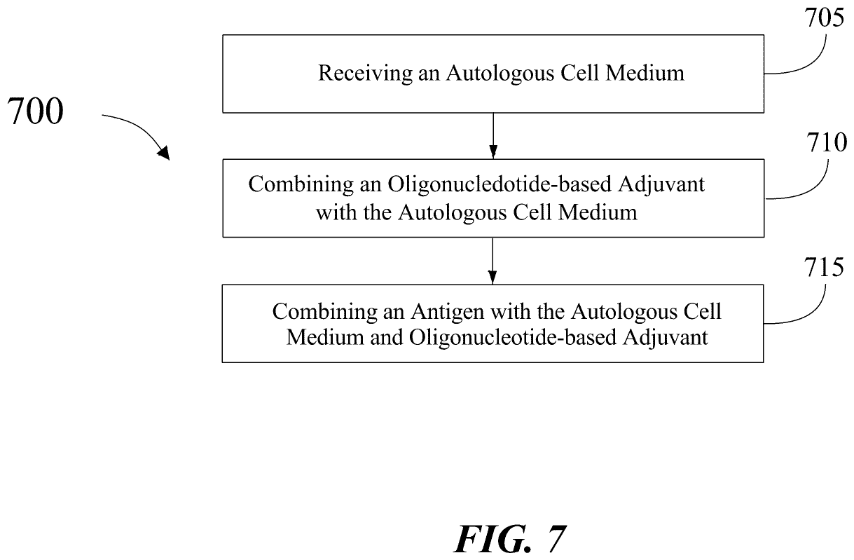

Referring now to FIG. 7, a method 700 of manufacturing a custom autologous vaccine is presented. At step 705, method 700 includes receiving an autologous cell medium. This step may be implemented as described above in FIGS. 1-6, without limitation.

Still referring to FIG. 7, at step 710, method 700 includes combining an oligonucleotide-based adjuvant with the autologous cell medium. This step may be implemented as described above in FIGS. 1-6, without limitation.

Still referring to FIG. 7, at step 715, method 700 includes combining an antigen with the autologous cell medium and oligonucleotide-based adjuvant. This step may be implemented as described above in FIGS. 1-6, without limitation.

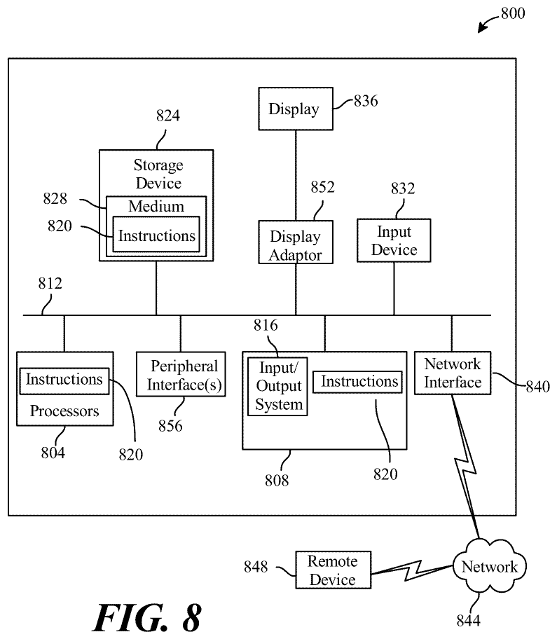

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components hereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of manufacturing a custom autologous vaccine, the method comprising:
receiving an autologous cell medium, wherein receiving the autologous cell medium further comprises producing the autologous cell medium using at least a cell collected from a subject, wherein the cell medium includes immune system stem cells and an allogeneic stem cell line;
combining an oligonucleotide-based adjuvant with the autologous cell medium;
synthesizing the oligonucleotide-based adjuvant with a second adjuvant; and
combining an antigen with the autologous cell medium and the oligonucleotide-based adjuvant.

2. The method of claim 1 further comprising extracting the immune system stem cells from the subject.

3. The method of claim 1, wherein the immune system stem cells comprise of fibroblast cells.

4. The method of claim 1, wherein the immune system stem cells comprise of retinal cells.

5. The method of claim 1, wherein the oligonucleotide-based adjuvant comprises an antisense oligonucleotide.

6. The method of claim 1, wherein the antigen comprises a nucleotide.

7. The method of claim 6, wherein the nucleotide further comprises mRNA.

8. The method of claim 1, wherein delivery of the vaccine comprises a targeting system to specific receptor sites.

9. The method of claim 1, further comprising generating a vaccine schedule.

10. The method of claim 9, wherein the vaccine schedule is customized to the subject based on biological factors.

11. The method of claim 10, wherein a biological factor comprises age.

12. The method of claim 1, wherein the antigen comprises a modified carrier virus.

13. The method of claim 1, wherein the oligonucleotide-based adjuvant is received from a user and purified in a cell line.

14. The method of claim 1, wherein the oligonucleotide-based adjuvant contains silver.

15. The method of claim 1, wherein the oligonucleotide-based adjuvant contains aluminum.

16. (canceled)

17. The method of claim 1, wherein the antigen comprises of a protein fragment of a bacterium.

18. The method of claim 1, wherein the immune system cells comprise of mast cells.

19. The method of claim 1, wherein the oligonucleotide-based adjuvant comprises a hexamer oligonucleotide.

20. The method of claim 1, wherein the immune system cells are cryopreserved.
